(19) United States
(12) Patent Application Publication
(10) Pub. No.: US 2012/0213329 A1
(43) Pub. Date: Aug. 23, 2012

(54) METAL TRANSPORT FRAME, BASE AND WORKSTATION FOR PANORAMIC DENTAL X-RAY MACHINE

(76) Inventor: Rodney Holum, JR., Onalaska, WI (US)

(21) Appl. No.: 13/031,507

(22) Filed: Feb. 21, 2011

(51) Int. Cl. A61B 6/14

(52) U.S. Cl. 378/38

(57) ABSTRACT

A combined transport frame, base, and workstation for holding a panoramic dental X-ray machine. A weighted metal base is structured with fork-lift receiving pallet-type openings. A hinge installed in the tall column enables the column to be pivoted down for shipping and storage to fit under normal doorways. High impact polyurethane panels are attached to the metal frame for shipping. Sides of frame open up with computer controls and printer. A double fan forced air cooling system in the X-ray head to keep the head cool allows many X-rays to be taken in a day.

### CROSS-REFERENCE TO RELATED APPLICATIONS

Not Applicable.

### STATEMENT REGARDING FEDERALLY SPONSORED RESEARCH OR DEVELOPMENT

Not Applicable.

### THE NAMES OF THE PARTIES TO A JOINT RESEARCH OR DEVELOPMENT Not Applicable.

### BACKGROUND OF THE INVENTION

1. Field of the Invention

The present invention relates to panoramic x-ray machines for dental x-rays and particularly to a combined transport frame, base and workstation for a holding a panoramic x-ray machine which comprises: a pallet-like weighted metal base for supporting a panoramic x-ray machine, the base having two openings therethrough for receiving the forks of a forklift; a protective metal frame seated on top of the base; computer controls for the x-ray machine installed in the frame sides, the right side (door) of frame pivots open for use as a printer stand, the left side (door) of frame pivots open for use as a computer stand; and x-ray head support bars with rubber pads on side frames which support X-ray machine head during transport.

2. Description of Related Art including information disclosed under 37 CFR 1.97 and 1.98

Transporting panoramic scanning dental X-ray machines presents a number of problems due to the weight, the instability caused by the weight being elevated on a column, and the height problem because the column is taller than the average doorway. Furthermore, the heat problem with the X-ray machines has generally limited the number of X-rays per day to five to ten X-rays per day. It is desirable to make the X-ray machines more portable to move them to a number of locations and perform large numbers of dental X-rays for large numbers of people each day from 30 to 200 people. Prior art devices have not solved these problems. U.S. Pat. No. 6,754,306, issued Jun. 22, 2004 to Cho et al, is for a portable medical digital radiography apparatus which includes a ruggedized transport case housing several system components, a digital X-ray image sensor and an X-ray generator. The digital X-ray image sensor is hingedly coupled to a stand that is mounted to the ruggedized transport case. The X-ray generator is preferably positioned opposite to the center of digital X-ray image sensor. A patient support unit is disposed between digital X-ray image sensor and the X-ray generator. An image acquisition unit is connected to the X-ray generator and to the digital X-ray image sensor and fixedly mounted in the ruggedized transport case. An image display unit is connected to the image acquisition unit and spaced from the X-ray generator.

U.S. Pat. No. 5,916,096, issued Jun. 29, 1999 to Weissmann et al, claims a portable care facility including at least one container defining a generally box-shaped configuration in a closed condition. The container is sized for transport and shipping and is constructed and arranged to be openable and expandable from the box-shaped configuration into a larger, operative configuration defining a space sufficient to conduct certain procedures therein. Tenting structure is provided in the container and is sized and configured to be supported over the space to define a ceiling over the space and to extend in at least one direction generally outwardly from the space to define a covered triage area.

U.S. Pat. No. 5,428,660, issued Jun. 27, 1995 to Daniel, Jr., shows a portable, lightweight medical radiographic device having an articulated support. The device includes a base, an elongated supporting structure having multiple sections interconnected by adjustable joints, a mount for a radiological camera, and a support for a film cartridge. The camera mount includes a concave, motorized track; the film cartridge support is also concave and may also provide motorized film advancement. A selected human body part to be imaged may be placed between the camera mount and the film track. When the camera, motorized track, and film support are energized, coordinated movement of the camera relative to the film results in a panoramic image of the body part on the exposed film.

Two U.S. Pat. No. 6,206,566 issued Mar. 27, 2001 to Schuetz and U.S. Pat. No. 6,379,041 issued Apr. 30, 2002 Schuetz et al, disclose an X-ray apparatus having an X-ray examination system with an X-ray source and an X-ray detector which can be displaced relative to a subject for the pickup of 2D projections, an arrangement for determining extrinsic and intrinsic imaging parameters, and having a control and computing means stage for reconstructing 3D images from the 2D projections using the extrinsic and intrinsic imaging parameters. The arrangement for determining the intrinsic imaging parameters includes X-ray-positive marks which are allocated to the X-ray source and which are substantially in one plane and in the path of an X-ray beam emanating from the X-ray source. U.S. Pat. No. 2,798,958, issued Jul. 9, 1957 to Hudson et al, provides a mobile apparatus for taking full-mouth X-rays.

U.S. Pat. No. 5,997,176, issued Dec. 7, 1999 to Fairleigh, claims an x-ray apparatus which produces a standard set of x-rays of a person's head while he or she is in a prone position. A movable frame is rigidly connected to a table, such as an operating table, and an extendible arm having a receiver for an x-ray source at a distal end provides for placement of the x-ray source in various, fixed positions. Right and left lateral cephalograms, an anterior-to-posterior cephalogram, and right and left transcranial views of the temporomandibular joint are provided by the fixed-position x-ray source. A panorama x-ray machine is also movably engaged with the frame for taking panographic images. A first set of cephalograms can be taken of a patient on an operating table, and the portable x-ray apparatus can be moved away from the operating table. A surgical treatment objective can be overlaid on the first set of cephalograms. Oral or orthognathic surgery or repair can be performed, and the portable x-ray apparatus can be subsequently reconnected to the operating table for taking a second set of cephalograms that precisely overlay the first set. A surgical team can thus compare actual operative results with the surgical treatment objective, and if necessary, make changes while the patient is still on the operating table.

U.S. Pat. No. 5,521,957, issued May 28, 1996 to Hansen, describes a portable X-ray imaging machine including a X-ray source and a X-ray image receptor. The source and the receptor are movable simultaneously along a line parallel to a patient reclined on the top of an examination table. The X-ray machine is readily transportable between and fixedly secured in a plurality of rooms in a hospital or other medical facility.

What is needed is means for transporting a dental X-ray device easily and on a regular basis fitting into normal buildings with normal door frame heights and to perform large numbers of dental X-rays at a time each day to serve large populations in a variety of locations.

### BRIEF SUMMARY OF THE INVENTION

The objectives of the present invention are to provide mobility and access for dental X-ray scanning machines by providing a base transportable by a fork lift to load it into a relatively small vehicle, providing a cut through the main column and sturdy hinge arrangement for the main column so that it can pivot down to fit through average doorways, providing dual fan air cooling for the X-ray heads to enable taking a large number of X-rays per day, and providing a sturdy metal frame with sides that pivot open with a built-in computer control and printer in the frame so that the dental X-ray scanning machines are easily transportable to any location and instantly set up to operate and serve large numbers of people each day.

In brief, the present invention comprises a combined transport frame, base, and workstation for holding a panoramic x-ray machine, particularly a KODAK® PANO X-ray machine for taking dental X-rays. The frame is closed around the PANO machine and high impact polyurethane panels are attached to the metal frame for shipping. The present invention includes a weighted metal base structured with fork-lift receiving pallet-type openings for use with a forklift to support a panoramic x-ray machine for transport and use. A hinge installed in the tall post enables the post to be pivoted down for shipping and storage to fit under normal doorways. Sides of frame open up with computer controls for PANO machine installed in sides of frame so it is instantly ready for use to X-ray patient's teeth with post pivoted up.

The left side (door) of frame pivots open for use as a computer stand to operate the PANO. The right side (door) of frame pivots open for use as a printer stand.

X-ray head support bars with rubber pads on side frames support the X-ray machine head during transport.

A special cooling system is attached to the X-ray machine head to keep the head cool to allow many X-rays to be taken in a day.

The support post is cut and hinged to allow the top of it to pivot down so metal shipping frame can be low enough to fit through regular doorways for installing the X-ray machine for use. The post is pivoted up to the full height for use to allow the X-ray head to ride on the post and be adjusted to different heights for different people. It is intended that each patient stand with the X-ray head surrounding the head of the patient to take a full set of X-rays of each patient's teeth very quickly and the X-rays are immediately saved and displayed as desired in the computer which is attached to one side frame. The X-ray results may also be printed out on the printer attached to the other side frame.

A special long bolt is used to secure the X-ray head from pivoting during transport.

An advantage of the present invention is it provides a combined base and pivotable frame structure to support and house the X-ray machine for both shipping and use.

Another advantage of the present invention is that it allows easy transport by pivoting down the support post to fit under standard seven foot high doorways.

### BRIEF DESCRIPTION OF THE SEVERAL VIEWS OF THE DRAWINGS

These and other details of my invention will be described in connection with the accompanying drawings, which are furnished only by way of illustration and not in limitation of the invention, and in which drawings:

FIG. 1 is a perspective view of a low profile transport frame, base, and work station system for a panoramic dental X-ray machine of the present invention shown with the sides open for use and the main column of the X-ray machine pivoted up for use;

FIG. 2 is a perspective view of a low profile transport frame, base, and work station system for a panoramic dental X-ray machine of FIG. 1 shown with the sides closed for transport and the main column of the X-ray machine pivoted down and showing a portion of a high impact polyurethane panel on the front of the frame;

FIG. 3 is a partial perspective view of the main column of the X-ray machine pivoted down and showing the hinge and a structural support piece installed inside the bottom of the column;

FIG. 4 is a perspective view of an X-ray head showing the dual fan cooling system of the present invention;

FIG. 5 is a partial perspective view of the top of the X-ray machine showing an elongated bolt protruding out of the top for easy access to secure the X-ray head from pivoting during transport.

### DETAILED DESCRIPTION OF THE INVENTION

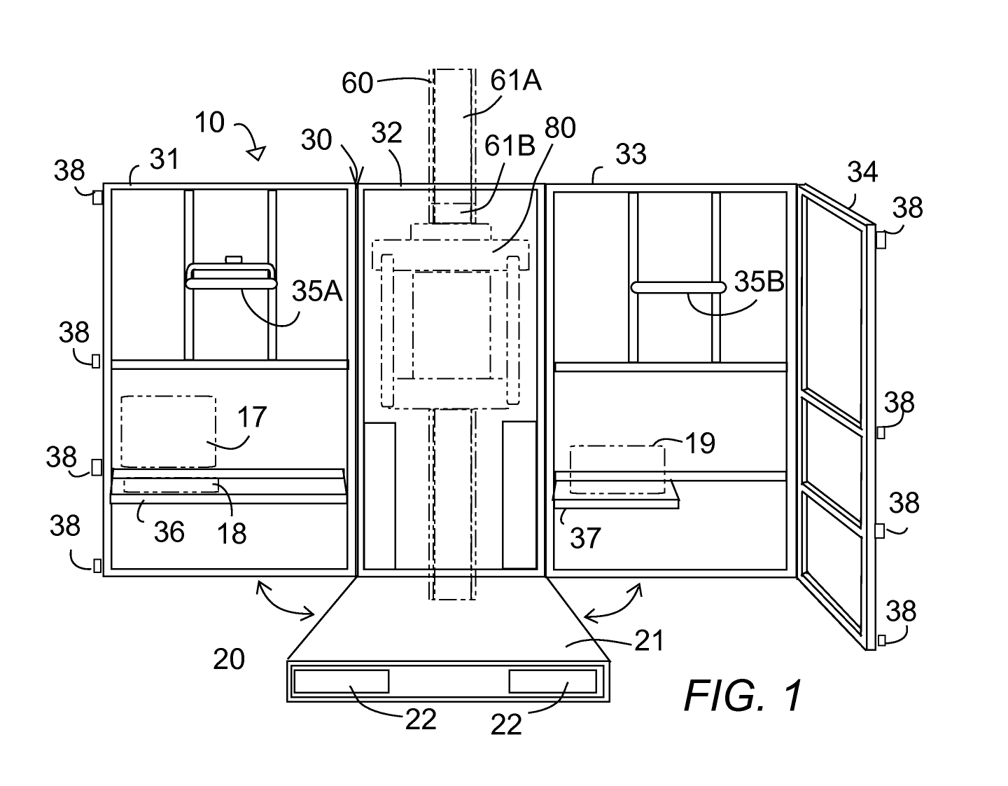

In FIGS. 1-5, a low profile transport system 10 for a panoramic dental X-ray machine provides a weighted base 20 to stabilize the top-heavy apparatus including openings 22 on the front and on the sides to receive a forklift or pallet jack for lift gate pick up in commercial buildings and for moving the device and a surrounding metal transport frame 30 having sides 31 and 33 which pivot open and provide an instant work station including a computer 18 and screen 17 and a printer 19 built into the frame for the panoramic dental X-ray machine, as shown in FIG. 1.

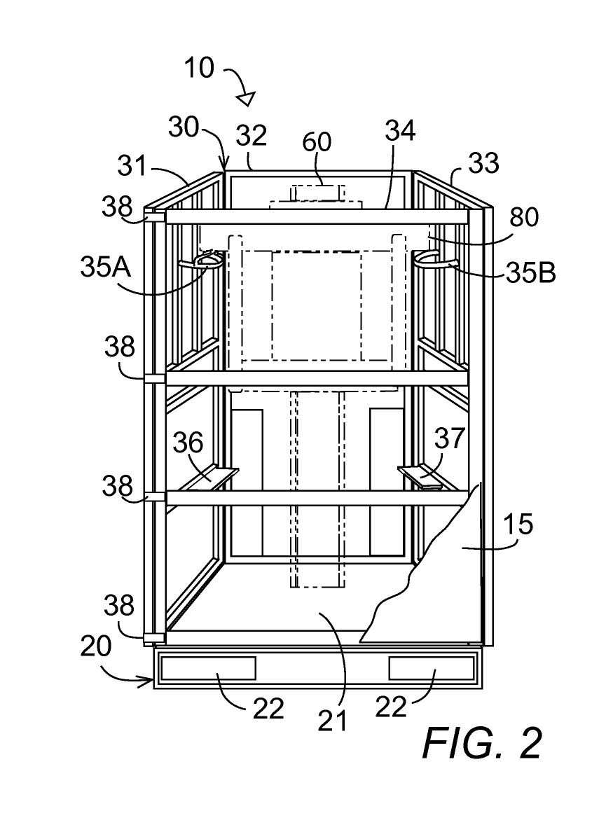

The rigid weighted support base 20 supports the dental X-ray machine body 80 and column 70 thereon. The base 20 is structured with openings 22 on the front, as shown in FIGS. 1 and 2, and also on the sides for a fork lift or pallet jack for lift gate pick up in commercial buildings to lift and move the dental X-ray machine on the support base, and the base is weighted to counterbalance the X-ray machine for a stable base during transport and use.

In FIG. 1, the rigid metal framework 30 is rigidly secured to the support base 20 and extends upwardly from the support base to surround and protect the dental X-ray machine body 80 during transport, as shown in FIG. 2. The framework comprises a vertical metal rear frame 32 attached to the support base by a permanent rigid connection, a pivotal vertical metal first side frame 31 hinged to a first vertical edge of the rear frame, and a pivotal vertical metal second side frame 33 hinged to a second vertical edge of the rear frame so that each of the side frames pivot between a first position at a right angle to the rear frame forming the side walls of the framework for transporting, as shown in FIG. 2, and a second position pivoted outwardly to form a linear alignment with the rear frame to permit access to the X-ray machine body 80 for use, as shown in FIG. 1. Side frame 31 has means, including the metal bracket with top padding 35A to support the X-ray machine body 80 thereon, as shown in FIG. 2, for supporting the X-ray machine in a stationary upright position in the first position for shipping and metal bracket 35B, on side frame 33, acts together with the metal bracket 35A to align the the X-ray machine body 80 to allow closing the two side frames 31 and 33. A rigid vertical front frame 34 permanently secured orthogonally to one of the side frames 33, so that the front frame secures to the other of the side frames 31 by latch locks 38 to lock the frame closed around the X-ray dental machine in the first position, as shown in FIG. 2. At least one work station, including the computer 18 on a computer support 36 on one pivotable side and a printer 19 on a printer stand 37 on the other pivotable side 33 are associated with operation of the dental X-ray machine in the second position of FIG. 1, to enable at least one user to work at the at least one work station.

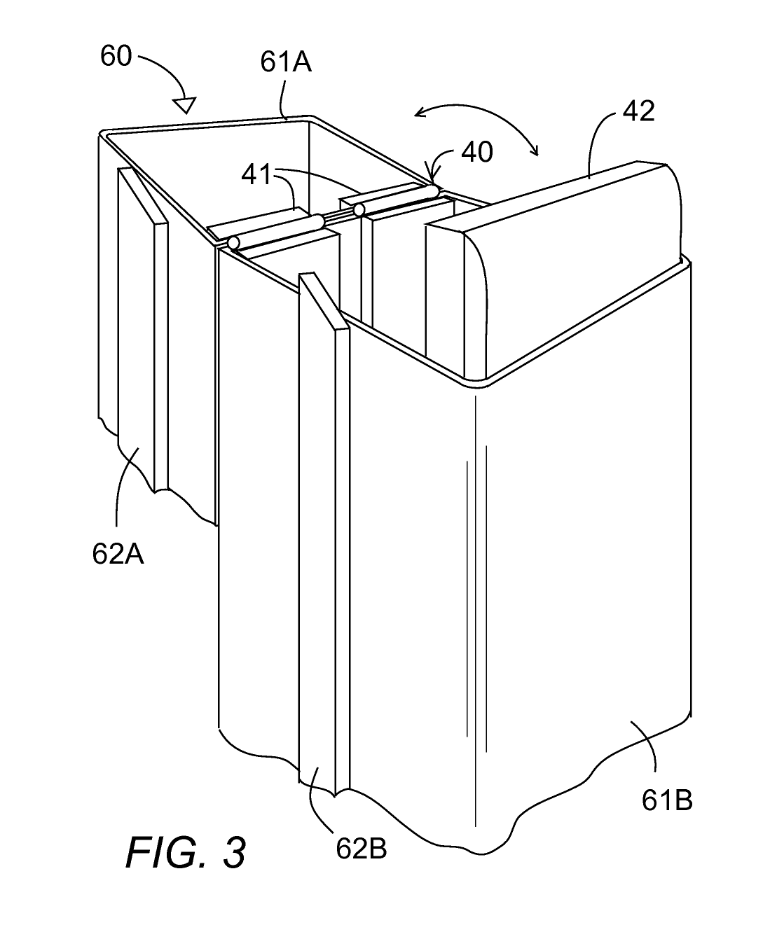

In FIG. 3, a hinge structure 40 is attached to a main support post 60 of the dental X-ray machine at a height lower than the rigid metal framework adjacent to a cut in the support post dividing the support post into a lower stationary portion 61B, lower than the framework structure to fit through a doorway, as shown in FIG. 2, and an upper pivoting portion 61A above the hinge structure so that the upper pivoting portion of the support post pivots back downwardly into a first position for transport outside the metal framework, as in FIG. 2, and pivots upwardly into linear alignment with the lower stationary portion 61B, for use to slide the X-ray machine body 80 vertically on the support post 60, as shown in FIG. 1, to a desired height for use with a patient standing adjacent to the support post.

High impact polyurethane panels 15, as shown in FIG. 2, are preferably attached to the metal framework 30 for support to further protect the X-ray machine during shipping.

The X-ray head support bars 36 and 37 on each of the side frames 31 and 33 support the at least one X-ray head 80, as shown in FIG. 2, to support and cushion the X-ray machine head 80 during transport.

In FIG. 5, an elongated bolt 50 through opening 53 secures the X-ray head 80 from pivoting during transport and is easily accessible for tightening for transport and loosening for use.

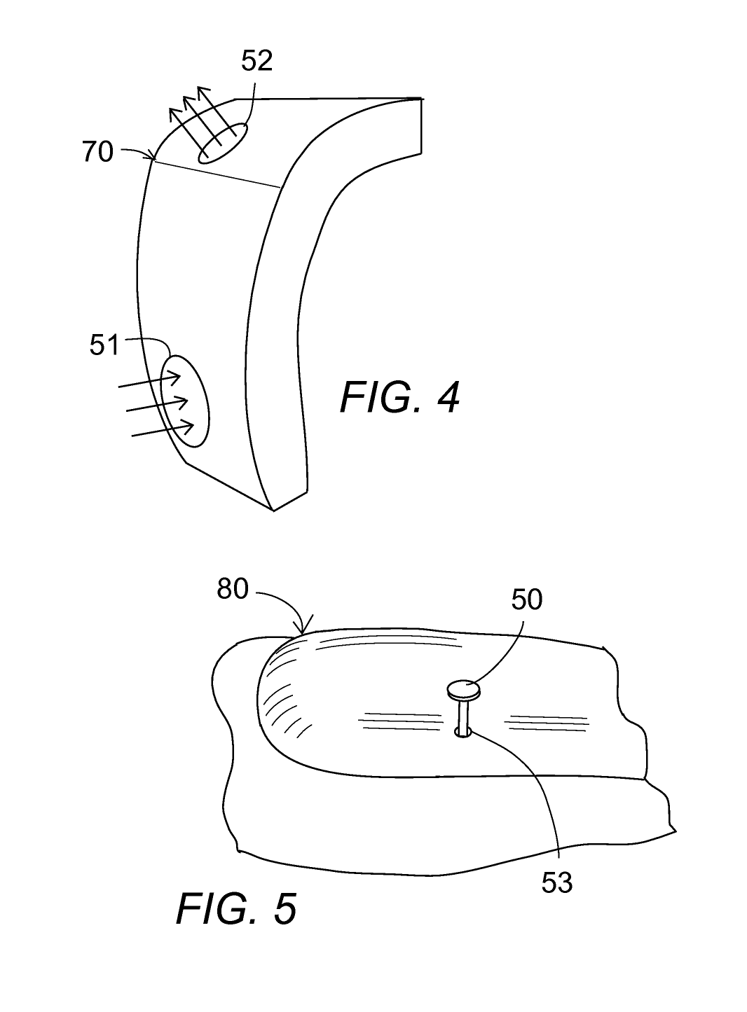

In FIG. 4, a double, including a forced air input fan 51 and a forced air output fan 52, provide an air circulating cooling system attached to the X-ray machine head 70 to keep the head cool to allow a plurality of X-rays to be taken in a day.

In use, the support post is cut and hinged to allow the top of it to pivot down so metal shipping frame can be low enough to fit through regular doorways for installing the X-ray machine for use. The post is pivoted up to the full height for use to allow the X-ray head to ride on the post and be adjusted to different heights for different people. It is intended that each patient stand with the X-ray head surrounding the head of the patient to take a full set of X-rays of each patients teeth very quickly and the X-rays are immediately saved and displayed as desired in the computer which is attached to one side frame. The X-ray results may also be printed out on the printer attached to the other side frame.

The weighted base plate provides a counter weight to the X-ray unit (which is preferably a KODAK® 8000 PANO).

It is intended that each patient stand with the X-ray head surrounding the head of the patient to take a panoramic x-ray of each patient very quickly and the X-rays are immediately saved and displayed as desired in the computer which is attached to one side frame. The X-ray results may also be printed out on the printer attached to the other side frame.

It is understood that the preceding description is given merely by way of illustration and not in limitation of the invention and that various modifications may be made thereto without departing from the spirit of the invention as claimed.

What is claimed is:

1. A low profile transport frame, base, and work station system for a panoramic dental X-ray machine, the system comprising:
a rigid support base to support a dental X-ray machine thereon, the base structured to receive a lifting arm of a fork lift therein to lift and move the dental X-ray machine on the support base, the base being weighted to counterbalance the X-ray machine for a stable base during transport and use;
a rigid metal framework rigidly secured to the support base and extending upwardly from the support base to surround and protect the dental X-ray machine during transport, the framework comprising a vertical metal rear frame attached to the support base by a permanent rigid connection; a pivotal vertical metal first side frame hinged to a first vertical edge of the rear frame and a pivotal vertical metal second side frame hinged to a second vertical edge of the rear frame so that each of the side frames pivot between a first position at a right angle to the rear frame forming the side walls of the framework for transporting and a second position pivoted outwardly to form a linear alignment with the rear frame to permit access to the X-ray machine for use, each of the side frames having means for supporting the X-ray machine in a stationary upright position in the first position; a rigid vertical front frame permanently secured orthogonally to one of the side frames so that the front frame secures to the other of the side frames to lock the frame closed around the X-ray dental machine in the first position; at least one work station associated with operation of the dental X-ray machine attached to at least one of the two vertical sides for operating the X-ray machine with the side frames in the second position to enable at least one user to work at the at least one work station;
a hinge structure attached to a main support post of the dental X-ray machine at a height lower than the rigid metal framework adjacent to a cut in the support post dividing the support post into a lower stationary portion lower than the framework structure to fit through a doorway, and an upper pivoting portion above the hinge structure so that the upper pivoting portion of the support post pivots downwardly into a first position for transport within the metal framework and pivots upwardly into linear alignment with the lower stationary portion for use to slide the X-ray head vertically on the support post to a desired height for use with a patient standing adjacent to the support post.

2. The system of claim 1 further comprising high impact polyurethane panels attached to the metal framework to further protect the X-ray machine during shipping.

3. The system of claim 1 wherein the at least one work station comprises a computer mounted on a computer stand on one of the side frames to operate the X-ray machine.

4. The system of claim 1 wherein the at least one work station comprises a printer mounted on a printer stand on one of the side frames to print out X-ray machine results.

5. The system of claim 1 further comprising at least one X-ray head support bar on each of the side frames, the at least one X-ray head support comprising a protruding metal frame having at least one upper rubber pad thereon to support and cushion the X-ray machine head during transport.

6. The system of claim 1 further comprising an elongated bolt to secure the X-ray head from pivoting during transport.

7. The system of claim 1 further comprising a double fan air circulating cooling system attached to the X-ray machine head to keep the head cool to allow a plurality of X-rays to be taken in a day.
